(19) United States
(12) Patent Application Publication
(10) Pub. No.: US 2007/0003624 A1
(43) Pub. Date: Jan. 4, 2007

(54) Semi-permeable compositions providing reduced drying time for osmotic dosage forms

(76) Inventors: Anant R. Shanbhag, Sunnyvale, CA (US); Brian L. Barclay, Sunnyvale, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/478,221

(22) Filed: Jun. 29, 2006

Related U.S. Application Data
(60) Provisional application No. 60/695,565, filed on Jun. 29, 2005.

(51) Int. Cl. A61K 9/28; A61K 9/24

(52) U.S. Cl. 424/473; 427/2.14

(57) ABSTRACT

Disclosed are methods including providing an osmotic core; coating the osmotic core with a coating composition that comprises cellulose acetate butyrate; drying the coated osmotic core for a maximum period of about 36 hours; wherein the coated core is dried to an average residual solvent content of less than about 1000 parts per million. Also disclosed are coated osmotic cores and methods of administering the coated osmotic cores to a patient.

### CROSS REFERENCE TO RELATED U.S. APPLICATIONS

The present application claims the benefit of Provisional application 60/695,565 filed Jun. 29, 2005.

### FIELD OF THE INVENTION

The invention relates to methods including providing an osmotic core; coating the osmotic core with a coating composition that comprises cellulose acetate butyrate; drying the coated osmotic core for a maximum period of about 36 hours; wherein the coated core is dried to an average residual solvent content of less than about 1000 parts per million. The invention further relates to coated osmotic cores and methods of administering the coated osmotic cores to a patient.

### BACKGROUND

Osmotic dosage forms in general utilize osmotic pressure to generate a driving force for imbibing fluid into a compartment formed, at least in part, by a semipermeable membrane that permits free diffusion of fluid but not drug or osmotic agent(s), if present. A significant advantage to osmotic systems is that operation is pH-independent and thus continues at the osmotically determined rate throughout an extended time period even as the dosage form transits the gastrointestinal tract and encounters differing microenvironments having significantly different pH values. A review of such dosage forms is found in Santus and Baker, “Osmotic drug delivery: a review of the patent literature,” Journal of Controlled Release 35 (1995) 1-21, incorporated by reference herein. U.S. Pat. Nos. 3,845,770; 3,916,899; 3,995,631; 4,008,719; 4,111,202; 4,160,020; 4,327,725; 4,578,075; 4,681,583; 5,019,397; and 5,156,850 disclose osmotic devices for the continuous dispensing of active agent.

Osmotic dosage forms in which a drug composition is delivered as a slurry, suspension or solution from a small exit orifice by the action of an expandable layer are disclosed in U.S. Pat. Nos. 5,633,011; 5,190,765; 5,252,338; 5,620,705; 4,931,285; 5,006,346; 5,024,842; and 5,160,743, which are incorporated herein by reference. Typical devices include an expandable push layer and a drug layer surrounded by a semipermeable membrane. In certain instances, the drug layer is provided with a subcoat to delay release of the drug composition to the environment of use or to form an annealed coating in conjunction with the semipermeable membrane.

A step in the manufacture of osmotic dosage forms is drying of the coated osmotic core to remove residual solvent left in the coated osmotic core from coating it with a semi-permeable membrane coating solution. Typically this step can be quite involved, especially for prior art semi-permeable membranes. Drying times can last for days and can require additional unit operations such as tray dryers with elevated temperature and humidity drying capability. While such conditions can hasten drying, labile drug compounds within the dosage form may be subject to degradation as a result of exposure to the more vigorous conditions. Moreover, extended drying times, 3-10 days being typical, can add to the overall production cost, and ultimately increases the cost of product to the patient

Accordingly, methods and compositions are needed that reduce drying requirements for coated osmotic cores in a manner that address the problems in the prior art noted above.

### BRIEF DESCRIPTION OF THE DRAWINGS

FIG. 1 shows an osmotic dosage form according to the invention.

FIG. 2 shows another osmotic dosage form according to the invention.

FIG. 3 shows a drying curve of dosage forms according to the invention.

FIG. 4 shows a cumulative release rate plot of dosage forms according to the invention.

FIG. 5 shows a cumulative release rate plot of dosage forms according to the invention.

FIG. 6 shows a cumulative release rate plot of dosage forms according to the invention.

### SUMMARY OF THE INVENTION

In an aspect, the invention relates to a method comprising: providing an osmotic core; coating the osmotic core with a coating composition that comprises cellulose acetate butyrate; drying the coated osmotic core for a maximum period of about 36 hours; wherein the coated core is dried to an average residual solvent content of less than about 1000 parts per million.

In another aspect, the invention relates to a method comprising: providing an osmotic core; coating the osmotic core with cellulose acetate butyrate; drying the coated osmotic core for a maximum period of about 24 hours; administering the coated osmotic core to a patient; wherein the coated core is dried to an average residual solvent content of less than about 1000 parts per million.

### DETAILED DESCRIPTION

Introduction

The inventors have unexpectedly discovered that the problems in the prior art can be addressed by the use of cellulose acetate butyrate in the semi-permeable membranes of osmotic dosage forms.

As will be discussed further herein, compared to conventional semi-permeable membrane compositions, the inventive semi-permeable membrane compositions provide for significantly reduced drying time, at lower cost and improved throughput. The present invention may also provide for improved product quality through reduction of heat-driven degradation of thermo-labile drugs.

Further, the relative permeability of the CAB based membrane is lower than that of CA membrane and can be modulated by varying the amount of the flux enhancer. Thus a much thinner CAB based membrane can achieve a permeability akin to CA-based membranes. This provides an additional advantage of reduced process time in the coating and drying processes.

The invention, and embodiments thereof, will now be described in more detail.

Definitions

All documents cited to herein are hereby incorporated by reference, for all purposes and in their entirety as if reproduced fully herein.

The present invention is best understood by reference to the following definitions, the drawings and exemplary disclosure provided herein.

“Osmotic core” means a formed composition that comprises at least one osmotically active substance and at least one orally deliverable drug (i.e. active agent) wherein the osmotic core is intended for use within an osmotic dosage form.

“Coating” means providing a film over a substrate.

“Coating composition” means a composition suitable for coating onto osmotic cores to form a semi-permeable membrane.

“Cellulose acetate butyrate” means a polymer that comprises esters of cellulose made by the action of a mixture of acetic and butyric acids and their anhydrides on purified cellulose, copolymers thereof, and equivalents thereof.

“Drying” means the removal of vaporized water or other liquid from a solid, liquid, or combination solid-liquid mixture (e.g. suspension) to promote generation of a dry solid. Generally, drying involves three transfer processes. The first process is heat transfer from an external source to the water or organic solvent in the material. The second process involves a phase change of the water or organic solvent from a liquid or liquid-like state to a vapor state. The third process is the mass transfer of the generated vapor away from the pharmaceutical material via the drying equipment. Drying may be accomplished in a variety of ways, including heating the material, reducing ambient pressure surrounding the material, and other conventional methods.

“Maximum period of about 24 hours” means a single continuous period that lasts no longer than about 24 hours.

“Dried to an average residual solvent content of less than about 1000 parts per million” means that the coated core is dried such that the solvent content of the coated core for a specific organic solvent used in the manufacture of the oral dosage form is less than about 1000 parts per million. Multiple organic solvents may be present in the dosage form; each of these may be present in an amount of less than about 1000 parts per million. The residual solvent(s) do not afford any therapeutic benefit and may adversely impact the aesthetic properties of the system due to its odor. Thus the residual solvent(s) in the system should be reduced to a minimum reasonable level. In a preferable embodiment, the coated osmotic cores are dried to an average residual solvent content of less than about 500 parts per million, even more preferably the coated osmotic cores are dried to an average residual solvent content of less than about 250 parts per million.

In an embodiment, the amount of residual solvent in the coated core is determined using a gas chromatographic method (GC) specific for the organic solvent of interest. In an embodiment, the organic solvent of interest comprises acetone, methanol, and/or ethanol. Sample composite solutions may be analyzed by a gas chromatographic system equipped with a flame ionization detector (FID) using columns selected from DB-WAX, Supelcowax-10, Stabilwax and HP-lnnowax. These columns have polyethylene glycol as the bonded phase. Quantitation may be performed by linear regression analysis of standard curves containing at least five standard points.

Reagents and supplies useful in the practice of the GC method comprise: an extraction solvent that comprises N,N-dimethylformamide, N-methylpyrrolidone Dimethyl acetamide(DMA), and water (These solvents are preferably reagent grade); Organic solvent reference standards (such as acetone, ethanol, methanol); Class A volumetric flasks and pipettes; GC liquid autosampler vials and caps; Stir bar and magnetic stirring plate; 5 cc plastic syringe; 25 mm syringe filter, 0.45 μm, GHP Acrodisc, Gelman or equivalent; Five-place analytical balance (Reading to 0.01 mg). The GC system preferably comprises a Gas Chromatograph: Hewlett Packard 6890 with EPC; or equivalent; Detector: Flame Ionization Detector (FID); Injector: Hewlett Packard 6890 Series Injector; or equivalent; Column: selected from those listed above. Operating parameters are preferably: Oven initial temp: 40° C.; Initial time: 0.1 min; Rate 1: 5° C./min; Final temp 1: 70° C.; Final time 1: 0 min; Rate 2: 50° C./min; Final temp 2: 230° C.; Final time 2: 5 min; Injector temp: 230° C.; Carrier gas: He at 5 mL/min (nominal); Split ratio: 20; Injection volume: 1 μL (2.0 μL with 5 μL sample syringe)→Syringe size may be varied but not the injection volume; Viscosity delay: 5 sec (nominal).

Detector temp: 230° C.; Air flow: 450 mL/min (nominal); H2 flow: 40 mL/min (nominal); Combined flow: 25 mL/min (makeup+column) (nominal); Makeup gas: N2.

A stock standard may be prepared as follows (acetone, methanol and ethanol will be used in the following discussion as example organic solvents that are to be assayed; one of skill in the art can modify this method to be applicable to other solvents): into 250 mL volumetric flask, add about 100 mL of extraction solvent; pipette 2 mL of acetone reference standard into the flask; pipette 1 mL of methanol reference standard into the flask; pipette 2 mL of ethanol reference standard into the flask; bring up to volume with extraction solvent, and cap tightly; mix well.

Working standards may be prepared as follows: prepare at least 5 working standards by making serial dilutions with extraction solvent as dilution solvent. Representative dilution scheme is shown below. Calibration curves should then be prepared.

| | | | [Ace- | | |
|---|---|---|---|---|---|
| Stand- | | Flask | tone]* | [Methanol]* | [Ethanol]* |
| ard | Volume | (mL) | (μg/mL) | (μg/mL) | (μg/mL) |
| Stock | 2 mL of acetone | 250 | 6328 | 3164 | 6352 |
| Std | 2 mL of ethanol | | | | |
| | 1 mL of methanol | | | | |
| Std-5 | 4 mL of stock Std | 250 | 101.2 | 50.62 | 101.6 |
| Std-4 | 2 mL of stock Std | 200 | 63.28 | 31.64 | 63.52 |
| Std-3 | 25 mL of Std-4 | 50 | 31.64 | 15.82 | 31.76 |
| Std-2 | 25 mL of Std-4 | 100 | 15.82 | 7.910 | 15.88 |
| Std-1 | 10 mL of Std-5 | 100 | 10.2 | 5.062 | 10.16 |

A Stock QC standard may be prepared as follows: into 250 mL volumetric flask, add about 100 mL of extraction solvent; pipette 2 mL of acetone reference standard into the flask; pipette 1 mL of methanol reference standard into the flask; pipette 2 mL of ethanol reference standard into the flask; bring up to volume with extraction solvent, and cap tightly; mix well. A working QC standard can be prepared as follows: make one dilution with extraction solvent as dilution solvent.

A representative dilution scheme is shown below.

| | | | [Ace- | | |
|---|---|---|---|---|---|
| Stand- | | Flask | tone]* | [Methanol]* | [Ethanol]* |
| ard | Volume | (mL) | (μg/mL) | (μg/mL) | (μg/mL) |
| Stock QC | 2 mL of acetone | 250 | 6328 | 3164 | 6352 |
| Std | 2 mL of ethanol | | | | |
| | 1 mL of methanol | | | | |
| QC Std | 2 mL of stock QC | 250 | 50.62 | 25.31 | 50.82 |

*Calculated based on purity factor of 100%
Note:
Stock standard solutions are stable up to 14 days under ambient condition.
Working standard solutions are stable up to 28 days under ambient condition.

In an embodiment, samples may be prepared as follows: weigh 5 coated cores and record the weight; place coated cores into 200 mL volumetric flask; pipette 100 mL of extraction solvent into 200 mL volumetric flask; place a stir bar into flask, cap and stir for at least 4 hours; filter sample solution using 0.45 μm filter and disposable 5 mL syringe; discard the first 2 mL solution; transfer an aliquot into GC vial for analysis. Sample solution for coated cores may be stable up to 3 days under ambient conditions.

System suitability may be tested follows: Inject a mid range standard six times. The system is suitable for analysis if the following criteria are met for all components: Area Response: RSD≦10%; Retention Time Variation: RSD≦5%; Tailing Factor (T): 0.5≦T≦3.5; Resolution: ≧1.5 (between peaks). Standards may be verified as follows: Inject extraction solvent blank prior to standard injections to ensure that the sum of all detected peaks within ±5% of the retention time of each interest peak should be ≦2% (area %) of the mid-range/target concentration level; establish a standard curve by injecting at least five working standards to bracket the expected sample concentration ranges. The calibration curves are acceptable if the correlation coefficient (r2) is ≧0.990. Calculated standards should be within ±15% of the original concentration for lowest standard, and within ±10% of the original concentration for other standards.

Samples may be analyzed as follows: Inject a QC standard prior to any sample analysis. The % recovery for the QC standard should be within ±10%. Periodically inject QC standard or mid range check standard.

The end of the analysis to check the system performance. If there are only two QC or mid range check standards, the % difference between the two standards should be within ±15%. If there are more than two QC or mid-range check standards, the % RSD should be ≦10%.

The stock standard concentration may be determined as follows: Determine the concentration of each solvent (for instance acetone, methanol and ethanol in the present exemplified embodiment) in the stock standard solution as follows:

$$Concentration ( µg / mL ) of solvent = \frac{( Density \times SV )}{TV} \times Purity$$

Where:

Density of methanol=791,000 μg/mL

Density of ethanol=794,000 μg/mL

Density of acetone=791,000 μg/mL

SV=Solvent volume of each component, mL

TV=Total volume, mL

Purity=Purity of reference standard

The amount of residual solvent in sample may be determined as follows: Determine the amount of each residual solvent in the samples from linear regression analysis of standard response versus concentration. Then calculate:

$$ppm ( µg / mL ) of solvent = \frac{( C \times V )}{W}$$

or

$$\% solvent = \frac{( C \times V )}{W} \times \frac{1 g}{1 \text{,} 000 \text{,} 000 µg} \times 100$$

where
C=Concentration of solvent via linear regression analysis, μg/mL
V=Volume of sample preparation, mL (e.g. 100 mL for 100 mg system or 50 mL for 25 mg systems)
W=Weight of 5 systems, g

“Dosage form” means a material suitable for pharmaceutical administration to a patient.

“Administering” means providing a material, especially a drug, to a patient.

“Patient” means a person or animal that is the object of study and/or medical intervention.

Semi-Permeable Membranes

Osmotic dosage forms according to the invention comprise semi-permeable membranes that surround an osmotic core. Such structures are discussed further elsewhere herein.

Materials useful for forming the semi-permeable membrane are essentially nonerodible and are substantially insoluble in biological fluids during the life of the dosage form. Representative polymers for forming the semi-permeable membrane comprise semipermeable homopolymers, semipermeable copolymers, and the like.

In an embodiment, the semi-permeable membrane according to the invention comprises cellulose acetate butyrate (“CAB”). Preferred grades of CAB include, but are not limited to: CAB-551-0.2, CAB-531-1, CAB-500.5, CAB-553-0.4, CAB-381-0.1, CAB-381-0.5, CAB-381-2, CAB-381-20, CAB-321-0.1, and CAB-171-15PG. Cellulose acetate butyrate may be obtained from the Eastman Chemical Company in a powder form. The nomenclature for the cellulose acetate butyrate as per the manufacturer Eastman Chemical Company is as follows. The first two digits indicate the butyryl content at the triester stage, the third digit indicates the number of hydroxyl units per four anhydroglucose units and the suffix indicates the viscosity in the solvent system designated by Eastman Chemical.

The semi-permeable membrane may also comprise an optional flux regulating agent. The flux regulating agent is a compound added to assist in regulating the fluid permeability or flux through the semi-permeable membrane. The flux regulating agent can be a flux enhancing agent or a decreasing agent. The agent can be preselected to increase or decrease the liquid flux. Agents that produce a marked increase in permeability to fluids such as water are often essentially hydrophilic, while those that produce a marked decrease to fluids such as water are essentially hydrophobic. The amount of regulator in the semi-permeable membrane when incorporated therein generally is from about 0.01% to about 40% by weight, preferably about 10 wt % to about 30 wt %, more preferably about 15 wt % to about 25 wt %, based on total solids in the coating composition.

The flux regulator agents in one embodiment that increase flux include, for example, polyhydric alcohols, polyalkylene glycols, polyalkylenediols, polyesters of alkylene glycols, and the like. Other pegylated compounds useful in the practice of the invention can be found in McCutheon's Detergents and Emulsifiers, International Edition, 1979 and BASF Pluronic and teronic surfactants 1999. and the like. Typical flux enhancers include polyethylene glycol 300, 400, 600, 1500, 4000, 6000, poly(ethylene glycol-co-propylene glycol), and the like; low molecular weight gylcols such as polypropylene glycol, polybutylene glycol and polyamylene glycol: the polyalkylenediols such as poly(1,3-propanediol), poly(1,4-butanediol), poly(1,6-hexanediol), and the like; aliphatic diols such as 1,3-butylene glycol, 1,4-pentamethylene glycol, 1,4-hexamethylene glycol, and the like; alkylene triols such as glycerine, 1,2,3-butanetriol, 1,2,4-hexanetriol, 1,3,6-hexanetriol and the like; esters such as ethylene glycol dipropionate, ethylene glycol butyrate, butylene glucol dipropionate, glycerol acetate esters, and the like.

Representative flux decreasing agents include, for example, phthalates substituted with an alkyl or alkoxy or with both an alkyl and alkoxy group such as diethyl phthalate, dimethoxyethyl phthalate, dimethyl phthalate, and [di(2-ethylhexyl)phthalate], aryl phthalates such as triphenyl phthalate, and butyl benzyl phthalate; insoluble salts such as calcium sulphate, barium sulphate, calcium phosphate, and the like; insoluble oxides such as titanium oxide; polymers in powder, granule and like form such as polystyrene, polymethylmethacrylate, polycarbonate, and polysulfone; esters such as citric acid esters esterfied with long chain alkyl groups; inert and substantially water impermeable fillers; resins compatible with cellulose based wall forming materials, and the like.

Other materials that can be used to form the semi-permeable membrane for imparting flexibility and elongation properties to the wall, for making the wall less-to-nonbrittle and to render tear strength, include, for example, phthalate plasticizers such as dibenzyl phthalate, dihexyl phthalate, butyl octyl phthalate, straight chain phthalates of six to eleven carbons, di-isononyl phthalte, di-isodecyl phthalate, and the like. The plasticizers include nonphthalates such as triacetin, dioctyl azelate, epoxidized tallate, tri-isoctyl trimellitate, tri-isononyl trimellitate, sucrose acetate isobutyrate, epoxidized soybean oil, and the like. The amount of plasticizer in a semi-permeable membrane when incorporated therein is about 0.01% to 20% weight, or higher. Other, conventionally known materials, such as antioxidants, colorants, stablizers, etc. may be added to the semi-permeable membrane.

The inventive semi-permeable membranes may be applied using techniques known in the art and/or disclosed elsewhere herein. Drying of the inventive semi-permeable membranes is disclosed elsewhere herein.

Dosage Forms

Osmotic dosage forms and methods of treatment using the osmotic dosage forms will now be described. It will be appreciated that the osmotic dosage forms described below are merely exemplary.

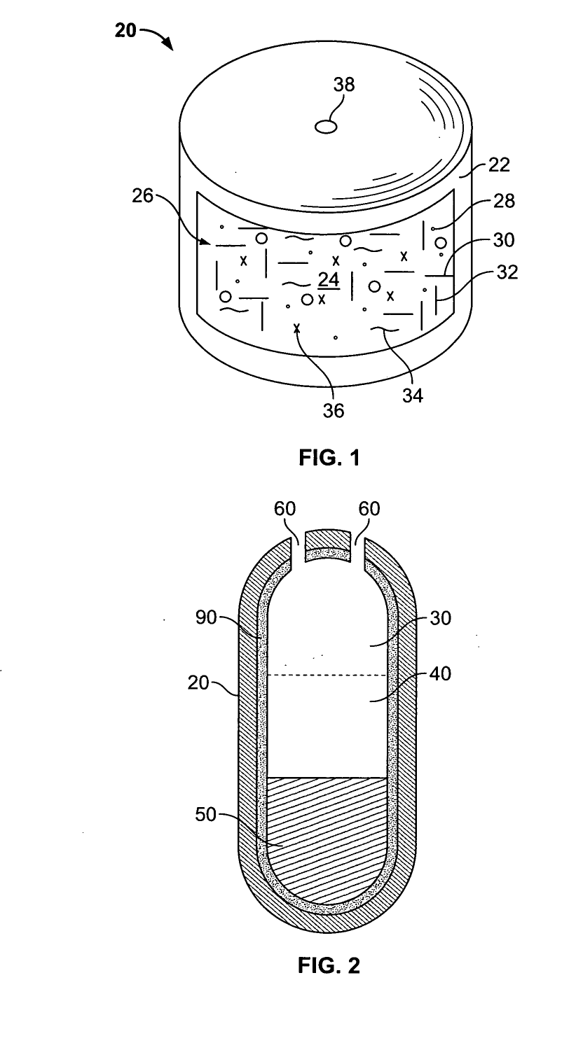

An exemplary osmotic dosage form, referred to in the art as an elementary osmotic pump dosage form, is shown in FIG. 1. Dosage form 20, shown in a cutaway view, is also referred to as an elementary osmotic pump, and is comprised of a semi-permeable wall 22 that surrounds and encloses an internal compartment 24. The internal compartment contains a single component layer referred to herein as a drug layer 26, comprising a drug 28 in an admixture with selected excipients. The excipients are adapted to provide an osmotic activity gradient for attracting fluid from an external environment through wall 22 and for forming a deliverable complex formulation upon imbibition of fluid. The excipients may include a suitable suspending agent, also referred to herein as drug carrier 30, a binder 32, a lubricant 34, and an osmotically active agent referred to as an osmagent 36. Exemplary materials useful for these components can be found disclosed throughout the present application.

Semi-permeable membrane 22 of the osmotic dosage form is permeable to the passage of an external fluid, such as water and biological fluids, but is substantially impermeable to the passage of components in the internal compartment. Materials, including CAB polymers, useful for forming the semi-permeable membrane have been discussed elsewhere.

In operation, the osmotic gradient across semi-permeable membrane 22 due to the presence of osmotically-active agents causes gastric fluid to be imbibed through the wall, swelling of the drug layer, and formation of a deliverable drug formulation (e.g., a solution, suspension, slurry or other flowable composition) within the internal compartment. The deliverable drug formulation is released through an exit 38 as fluid continues to enter the internal compartment. Even as drug formulation is released from the dosage form, fluid continues to be drawn into the internal compartment, thereby driving continued release. In this manner, the drug is released in a sustained and continuous manner over an extended time period.

FIG. 2 illustrates certain inventive embodiments of sustained release dosage forms. Dosage forms of this type are described in detail in U.S. Pat. Nos. 4,612,008; 5,082,668; and 5,091,190; and are further described below

FIG. 2 shows an embodiment of one type of sustained release dosage form, namely the osmotic sustained release dosage form. First drug layer 30 comprises osmotically active components, and a lower amount of active agent than in second drug layer 40. The osmotically active component(s) in the first component drug layer comprises an osmagent such as salt and one or more osmopolymer(s) having relatively small molecular weights which exhibit swelling as fluid is imbibed such that release of these osmopolymers through exit 60 occurs similar to that of drug layer 40. Additional excipients such as binders, lubricants, antioxidants and colorants may also be included in first drug layer 30.

Second drug layer 40 comprises active agent in an admixture with selected excipients adapted to provide an osmotic activity gradient for driving fluid from an external environment through semi-permeable membrane 20 and for forming a deliverable drug formulation upon imbibition of fluid. The excipients may include a suitable suspending agent, also referred to herein as a drug carrier, but no osmotically active agent, “osmagent,” such as salt, sodium chloride. It has been discovered that the omission of salt from this second drug layer, which contains a higher proportion of the overall drug in the dosage form, in combination with the salt in the first drug layer, provides an improved ascending rate of release creating a longer duration of ascending rate.

Drug layer 40 has a higher concentration of the drug than does drug layer 30. The ratio of the concentration of drug in the first drug layer 30 to the concentration of drug in the second drug layer 40 is maintained at less than 1 and preferably less than or equal to about 0.43 to provide the desired substantially ascending rate of release.

Drug layer 40 may also comprise other excipients such as lubricants, binders, etc.

Drug layer 40, as with drug layer 30, further comprises a hydrophilic polymer carrier. The hydrophilic polymer provides a particle in the drug composition that contributes to the controlled delivery of the active drug. Representative examples of these polymers are poly(alkylene oxide) of 100,000 to 750,000 number-average molecular weight, including poly(ethylene oxide), poly(methylene oxide), poly(butylene oxide) and poly(hexylene oxide); and a poly(carboxymethylcellulose) of 40,000 to 400,000 number-average molecular weight, represented by poly(alkali carboxymethylcellulose), poly(sodium carboxymethylcellulose), poly(potassium carboxymethylcellulose) and poly(lithium carboxymethylcellulose). Drug layer 40 can further comprise a hydroxypropylalkylcellulose of 9,200 to 125,000 number-average molecular weight for enhancing the delivery properties of the dosage form as represented by hydroxypropylethylcellulose, hydroxypropylmethylcellulose, hydroxypropylbutylcellulose and hydroxypropylpentylcellulose; and a poly(vinylpyrrolidone) of 7,000 to 75,000 number-average molecular weight for enhancing the flow properties of the dosage form. Preferred among these polymers are the poly(ethylene oxide) of 100,000 -300,000 number average molecular weight. Carriers that erode in the gastric environment, i.e., bioerodible carriers, are especially preferred.

Other carriers that may be incorporated into drug layer 40, and/or drug layer 30, include carbohydrates that exhibit sufficient osmotic activity to be used alone or with other osmagents. Such carbohydrates comprise monosaccharides, disaccharides and polysaccharides. Representative examples include maltodextrins (i.e., glucose polymers produced by the hydrolysis of corn starch) and the sugars comprising lactose, glucose, raffinose, sucrose, mannitol, sorbitol, and the like. Preferred maltodextrins are those having a dextrose equivalence (DE) of 20 or less, preferably with a DE ranging from about 4 to about 20, and often 9-20. Maltodextrin having a DE of 9-12 has been found to be useful.

Drug layer 40 and drug layer 30 typically will be a substantially dry, <1% water by weight, composition formed by compression of the carrier, the drug, and other excipients as one layer.

Drug layer 40 may be formed from particles by comminution that produces the size of the drug and the size of the accompanying polymer used in the fabrication of the drug layer, typically as a core containing the compound, according to the mode and the manner of the invention. The means for producing particles include granulation, spray drying, sieving, lyophilization, crushing, grinding, jet milling, micronizing and chopping to produce the intended micron particle size. The process can be performed by size reduction equipment, such as a micropulverizer mill, a fluid energy grinding mill, a grinding mill, a roller mill, a hammer mill, an attrition mill, a chaser mill, a ball mill, a vibrating ball mill, an impact pulverizer mill, a centrifugal pulverizer, a coarse crusher and a fine crusher. The size of the particle can be ascertained by screening, including a grizzly screen, a flat screen, a vibrating screen, a revolving screen, a shaking screen, an oscillating screen and a reciprocating screen. The processes and equipment for preparing drug and carrier particles are disclosed in Pharmaceutical Sciences, Remington, 17th Ed., pp. 1585-1594 (1985); Chemical Engineers Handbook, Perry, 6th Ed., pp. 21-13 to 21-19 (1984); Journal of Pharmaceutical Sciences, Parrot, Vol. 61, No. 6, pp. 813-829 (1974); and Chemical Engineer, Hixon, pp. 94-103 (1990).

First drug layer 30 comprises active agent in an admixture with selected excipients adapted to provide an osmotic activity gradient for driving fluid from an external environment through semi-permeable membrane 20 and for forming a deliverable drug formulation upon imbibition of fluid. The excipients may include a suitable suspending agent, also referred to herein as a drug carrier, and an osmotically active agent, i.e., an “osmagent,” such as salt. Other excipients such as lubricants, binders, etc. may also be included.

The osmotically active component in the first drug layer typically comprises an osmagent and one or more osmopolymer(s) having relatively small molecular weights which exhibit swelling as fluid is imbibed such that release of these osmopolymers through exit 60 occurs similar to that of drug layer 40.

The ratio of drug concentration between the first drug layer and the second drug layer alters the release rate profile. Release rate profile is calculated as the difference between the maximum release rate and the release rate achieved at the first time point after start-up (for example, at 6 hours), divided by the average release rate between the two data points.

Drug layer 30 and drug layer 40 may optionally contain surfactants and disintegrants in both drug layers. Exemplary of the surfactants are those having an HLB value of about 10-25, such as polyethylene glycol 400 monostearate, polyoxyethylene-4-sorbitan monolaurate, polyoxyethylene-20-sorbitan monooleate, polyoxyethylene-20-sorbitan monopalmitate, polyoxyethylene-20-monolaurate, polyoxyethylene-40-stearate, sodium oleate and the like.

Disintegrants may be selected from starches, clays, celluloses, alginates and gums and crosslinked starches, celluloses and polymers. Representative disintegrants include corn starch, potato starch, croscarmelose, crospovidone, sodium starch glycolate, Veegum HV, methylcellulose, agar, bentonite, carboxymethylcellulose, alginic acid, guar gum and the like.

Semipermeable membrane 20 is formed to be permeable to the passage of an external fluid, such as water and biological fluids, and is substantially impermeable to the passage of active agent, osmagent, osmopolymer and the like. As such, it is semipermeable. The selectively semipermeable CAB compositions used for forming semi-permeable membrane 20 have been discussed elsewhere herein.

Push layer 50 comprises an expandable layer in contacting layered arrangement with the second component drug layer 40 as illustrated in FIG. 2. Push layer 50 comprises a polymer that imbibes an aqueous or biological fluid and swells to push the drug composition through the exit of the device.

The expandable layer comprises in one embodiment a hydroactivated composition that swells in the presence of water, such as that present in gastric fluids. Conveniently, it can comprise an osmotic composition comprising an osmotic solute that exhibits an osmotic pressure gradient across the semipermeable layer against an external fluid present in the environment of use. In another embodiment, the hydro-activated layer comprises a hydrogel that imbibes and/or absorbs fluid into the layer through the outer semipermeable wall. The semipermeable wall is non-toxic. It maintains its physical and chemical integrity during operation and it is essentially free of interaction with the expandable layer.

The expandable layer in one preferred embodiment comprises a hydroactive layer comprising a hydrophilic polymer, also known as osmopolymers. The osmopolymers exhibit fluid imbibition properties. The osmopolymers are swellable, hydrophilic polymers, which osmopolymers interact with water and biological aqueous fluids and swell or expand to an equilibrium state. The osmopolymers exhibit the ability to swell in water and biological fluids and retain a significant portion of the imbibed fluid within the polymer structure. The osmopolymers swell or expand to a very high degree, usually exhibiting a 2 to 50 fold volume increase. The osmopolymers can be non-cross-linked or cross-linked. The swellable, hydrophilic polymers are in one embodiment lightly cross-linked, such cross-links being formed by covalent or ionic bonds or residue crystalline regions after swelling. The osmopolymers can be of plant, animal or synthetic origin.

The osmopolymers are hydrophilic polymers. Hydrophilic polymers suitable for the present purpose include poly(hydroxy-alkyl methacrylate) having a molecular weight of from 30,000 to 5,000,000; poly(vinylpyrrolidone) having a molecular weight of from 10,000 to 360,000; anionic and cationic hydrogels; polyelectrolytes complexes; poly(vinyl alcohol) having a low acetate residual, cross-linked with glyoxal, formaldehyde, or glutaraldehyde and having a degree of polymerization of from 200 to 30,000; a mixture of methyl cellulose, cross-linked agar and carboxymethyl cellulose; a mixture of hydroxypropyl methylcellulose and sodium carboxymethylcellulose; a mixture of hydroxypropyl ethylcellulose and sodium carboxymethyl cellulose, a mixture of sodium carboxymethylcellulose and methylcellulose, sodium carboxymethylcellulose; potassium carboxymethylcellulose; a water insoluble, water swellable copolymer formed from a dispersion of finely divided copolymer of maleic anhydride with styrene, ethylene, propylene, butylene or isobutylene crosslinked with from 0.001 to about 0.5 moles of saturated cross-linking agent per mole of maleic anhydride per copolymer; water swellable polymers of N-vinyl lactams; polyoxyethylene-polyoxypropylene gel; carob gum; polyacrylic gel; polyester gel; polyuria gel; polyether gel, polyamide gel; polycellulosic gel; polygum gel; initially dry hydrogels that imbibe and absorb water which penetrates the glassy hydrogel and lowers its glass temperature; and the like.

Representative of other osmopolymers are polymers that form hydrogels such as Carbopol™. acidic carboxypolymer, a polymer of acrylic acid cross-linked with a polyallyl sucrose, also known as carboxypolymethylene, and carboxyvinyl polymer having a molecular weight of 250,000 to 4,000,000; Cyanamer™ polyacrylamides; cross-linked water swellable indenemaleic anhydride polymers; Good-rite™ polyacrylic acid having a molecular weight of 80,000 to 200,000; Polyox™ polyethylene oxide polymer having a molecular weight of 100,000 to 5,000,000 and higher; starch graft copolymers; Aqua-Keeps™ acrylate polymer polysaccharides composed of condensed glucose units such as diester cross-linked polygluran; and the like. Representative polymers that form hydrogels are known to the prior art in U.S. Pat. No. 3,865,108; U.S. Pat. No. 4,002,173; U.S. Pat. No. 4,207,893; and in Handbook of Common Polymers, by Scott and Roff, published by the Chemical Rubber Co., Cleveland, Ohio. The amount of osmopolymer comprising a hydro-activated layer can be from about 5% to 100%.

The expandable layer in another manufacture can comprise an osmotically effective compound that comprises inorganic and organic compounds that exhibit an osmotic pressure gradient across a semipermeable wall against an external fluid. The osmotically effective compounds, as with the osmopolymers, imbibe fluid into the osmotic system, thereby making available fluid to push against the inner wall, i.e., in some embodiments, the barrier layer and/or the wall of the soft or hard capsule for pushing active agent from the dosage form. The osmotically effective compounds are known also as osmotically effective solutes, and also as osmagents. Osmotically effective solutes that can be used comprise magnesium sulfate, magnesium chloride, potassium sulfate, sodium sulfate, lithium sulfate, potassium acid phosphate, mannitol, urea, inositol, magnesium succinate, tartaric acid, carbohydrates such as raffinose, sucrose, glucose, lactose, sorbitol, and mixtures therefor. The amount of osmagent in can be from about 5% to 100% of the weight of the layer. The expandable layer optionally comprises an osmopolymer and an osmagent with the total amount of osmopolymer and osmagent equal to 100%. Osmotically effective solutes are known to the prior art as described in U.S. Pat. No. 4,783,337.

Inner wall 90 further provides a lubricating function that facilitates the movement of first drug layer 30, second drug layer 40 and push layer 50 toward exit 60. Inner wall 90 may be formed from hydrophilic materials and excipients. Semipermeable membrane 20 is semipermeable, allowing gastric fluid to enter the compartment, but preventing the passage of the materials comprising the core in the compartment. The deliverable drug formulation is released from exit 60 upon osmotic operation of the osmotic oral dosage form.

Inner wall 90 also reduces friction between the external surface of drug layer 30 and drug layer 40, and the inner surface of semipermeable membrane 20. Inner wall 90 promotes release of the drug composition from the compartment and reduces the amount of residual drug composition remaining in the compartment at the end of the delivery period, particularly when the slurry, suspension or solution of the drug composition that is being dispensed is highly viscous during the period of time in which it is being dispensed. In dosage forms with hydrophobic agents and no inner wall, it has been observed that significant residual amounts of drug may remain in the device after the period of delivery has been completed. In some instances, amounts of 20% or greater may remain in the dosage form at the end of a twenty-four hour period when tested in a release rate assay.

Inner wall 90 is formed as an inner coat of a flow-promoting agent, i.e., an agent that lowers the frictional force between the semi-permeable membrane 20 and the external surface of drug layer 40. Inner wall 90 appears to reduce the frictional forces between semi-permeable membrane 20 and the outer surface of drug layer 30 and drug layer 40, thus allowing for more complete delivery of drug from the device. Particularly in the case of active compounds having a high cost, such an improvement presents substantial economic advantages since it is not necessary to load the drug layer with an excess of drug to insure that the minimum amount of drug required will be delivered. Inner wall 90 may be formed as a coating applied over the compressed core.

Inner wall 90 typically may be 0.01 to 5 mm thick, more typically 0.5 to 5 mm thick, and it comprises a member selected from hydrogels, gelatin, low molecular weight polyethylene oxides, e.g., less than 100,000 MW, hydroxyalkylcelluloses, e.g., hydroxyethylcellulose, hydroxypropylcellulose, hydroxyisopropylcelluose, hydroxybutylcellulose and hydroxyphenylcellulose, and hydroxyalkyl alkylcelluloses, e.g., hydroxypropyl methylcellulose, and mixtures thereof. The hydroxyalkylcelluloses comprise polymers having a 9,500 to 1,250,000 number-average molecular weight. For example, hydroxypropyl celluloses having number average molecular weights of 80,000 to 850,000 are useful. The inner wall may be prepared from conventional solutions or suspensions of the aforementioned materials in aqueous solvents or inert organic solvents.

Prefered materials for the inner wall include hydroxypropyl cellulose, hydroxyethyl cellulose, hydroxypropyl methyl cellulose, povidone [poly(vinylpyrrolidone)], polyethylene glycol, and mixtures thereof.

Most prefered are mixtures of hydroxypropyl cellulose and povidone, prepared in organic solvents, particularly organic polar solvents such as lower alkanols having 1-8 carbon atoms, preferably ethanol, mixtures of hydroxyethyl cellolose and hydroxypropyl methyl cellulose prepared in aqueous solution, and mixtures of hydroxyethyl cellulose and polyethylene glycol prepared in aqueous solution. Most preferably, the inner wall comprises a mixture of hydroxypropyl cellulose and providone prepared in ethanol.

It is preferred that inner wall 90 comprises between about 50% and about 90% hydroxypropylcellulose identified as EF having an average molecular weight of about 80,000 and between about 10% and about 50% polyvinylpyrrolidone identified as K29-32.

Conveniently, the weight of the inner wall applied to the compressed core may be correlated with the thickness of the inner wall and residual drug remaining in a dosage form in a release rate assay such as described herein. As such, during manufacturing operations, the thickness of the inner wall may be controlled by controlling the weight of the inner wall taken up in the coating operation.

When inner wall 90 is formed as a subcoat, i.e., by coating onto the tabletted composite including one or all of the first drug layer, second drug layer and push layer, the inner wall can fill in surface irregularities formed on the core by the tabletting process. The resulting smooth external surface facilitates slippage between the coated composite core and the semipermeable wall during dispensing of the drug, resulting in a lower amount of residual drug composition remaining in the device at the end of the dosing period. When inner wall 90 is fabricated of a gel-forming material, contact with water in the environment of use facilitates formation of the gel or gel-like inner coat having a viscosity that may promote and enhance slippage between semi-permeable membrane 20 and drug layer 30 and drug layer 40.

Pan coating may be conveniently used to provide the completed dosage form, except for the exit orifice. In the pan coating system, the wall-forming composition for the inner wall or the outer wall, as the case may be, is deposited by successive spraying of the appropriate wall composition onto the compressed trilayered or multilayered core comprising the drug layers, optional barrier layer and push layer, accompanied by tumbling in a rotating pan. A pan coater is used because of its availability at commercial scale. Other techniques can be used for coating the compressed core. Once coated, the wall is dried in a forced-air oven or in a temperature and humidity controlled oven to free the dosage form of solvent(s) used in the manufacturing. Drying conditions will be conventionally chosen on the basis of available equipment, ambient conditions, solvents, coatings, coating thickness, and the like.

Other coating techniques can also be employed. For example, the wall or walls of the dosage form may be formed in one technique using the air-suspension procedure. This procedure consists of suspending and tumbling the compressed core in a current of air and the semipermeable wall forming composition, until the wall is applied to the core. The air-suspension procedure is well suited for independently forming the wall of the dosage form. The air-suspension procedure is described in U.S. Pat. No. 2,799,241; in J. Am. Pharm. Assoc., Vol. 48, pp. 451459 (1959); and, ibid., Vol. 49, pp. 82-84 (1960). The dosage form also can be coated with a Wurster® air-suspension coater using, for example, methylene dichloride methanol as a cosolvent for the wall forming material. An Aeromatic® air-suspension coater can be used employing a cosolvent.

In an embodiment, the sustained release dosage form of the invention is provided with at least one exit 60 as shown in FIG. 2. Exit 60 cooperates with the compressed core for the uniform release of drug from the dosage form. The exit can be provided during the manufacture of the dosage form or during drug delivery by the dosage form in a fluid environment of use.

One or more exit orifices are drilled in the drug layer end of the dosage form, and optional water soluble overcoats, which may be colored (e.g., Opadry colored coatings) or clear (e.g., Opadry Clear), may be coated on the dosage form to provide the finished dosage form.

Exit 60 may include an orifice that is formed or formable from a substance or polymer that erodes, dissolves or is leached from the outer wall to thereby form an exit orifice. The substance or polymer may include, for example, an erodible poly(glycolic) acid or poly(lactic) acid in the semipermeable wall; a gelatinous filament; a water-removable poly(vinyl alcohol); a leachable compound, such as a fluid removable pore-former selected from the group consisting of inorganic and organic salt, oxide and carbohydrate.

An exit, or a plurality of exits, can be formed by leaching a member selected from the group consisting of sorbitol, lactose, fructose, glucose, mannose, galactose, talose, sodium chloride, potassium chloride, sodium citrate and mannitol to provide a uniform-release dimensioned pore-exit orifice.

The exit can have any shape, such as round, triangular, square, elliptical and the like for the uniform metered dose release of a drug from the dosage form.

The sustained release dosage form can be constructed with one or more exits in spaced-apart relation or one or more surfaces of the sustained release dosage form.

Drilling, including mechanical and laser drilling, through the semipermeable wall can be used to form the exit orifice. Such exits and equipment for forming such exits are disclosed in U.S. Pat. No. 3,916,899, by Theeuwes and Higuchi and in U.S. Pat. No. 4,088,864, by Theeuwes, et al. It is presently preferred to utilize two exits of equal diameter. In a preferred embodiment, exit 60 penetrates through subcoat 90, if present, to drug layer 30.

In another embodiment, the drug and other ingredients comprising a therapeutic composition or comprising the drug layer facing the exit are blended, or they are blended then pressed, into a solid layer. The drug and other ingredients can be blended with a solvent and formed into a solid or semisolid formed by conventional methods such as ball-milling, calendering, stirring or roll-milling and then pressed into a selected shape. The layer possesses dimensions that correspond to the internal dimensions of the area the layer is to occupy in the dosage form. The bilayer possesses dimensions corresponding to the internal lumen of the dosage form. Next, a hydrogel “push-layer” is placed in contact with the drug layer. The layering of the drug layer and the hydrogel push-layer can be fabricated by conventional press-layering techniques. Finally, the two-layer compartment forming members are surrounded and coated with an outer wall. A passageway is laser drilled or mechanically drilled through the wall to contact the drug layer, with the dosage form optically oriented automatically by the laser equipment for forming the passageway on the preselected drug surface.

In another embodiment, the dosage form is manufactured by the wet granulation technique. In the wet granulation technique, the drug and the ingredients comprising the first layer are blended using an organic or inorganic solvent, such as isopropyl alcohol-methylene dichloride 80:20 (v:v) as the granulation fluid. Other granulating fluid, such as water, isopropyl alcohol, or denatured alcohol 100% can be used for this purpose. The ingredients forming the drug layer are individually passed through a 40-mesh screen, then thoroughly blended in a mixer. Next, other ingredients comprising the drug layer are dissolved in a portion of the granulation fluid, such as the cosolvent described above. Then, the latter prepared wet blend is slowly added to the drug blend with continual mixing in the blender. The granulating fluid is added until a wet blend mass is produced, which wet mass is then forced through a 20-mesh screen onto oven trays. The blend is dried for 18 to 24 hours at 25° C. to 40° C. The dry granules are then screened with a 16-mesh screen. Next, a lubricant is passed through a 60-mesh screen and added to the dry screened granule blend. The granulation is put into milling jars and mixed on a jar mill for 2 to 10 minutes. The drug layer and push-layer compositions are pressed into a layered tablet, for example, on a Manesty® layer press.

Another manufacturing process that can be used for providing the drug and hydrogel composition comprises blending their powdered ingredients in a fluid-bed granulator. After the powdered ingredients are dry blended in the granulator, a granulating fluid, for example, poly(vinylpyrrolidone) in a solvent, such as in water, is sprayed onto the respective powders. The coated powders are then dried in the granulator. This process coats the dry ingredients present therein while spraying the granulating fluid. After the granules are dried, a lubricant, such as stearic acid or magnesium stearate, is blended as above into the mixture. The granules are then pressed in the manner described above. In another embodiment, when the fluid-bed granulating process is used to manufacture the hydrogel layer, the antioxidant present in the polyalkylene oxide can be removed during the processing step. If antioxidant is desired, it can be added to the hydrogel formulation; this can be accomplished during the fluid-bed granulation described above.

Dosage forms according to the invention may be manufactured in another embodiment by mixing the drug with composition-forming ingredients and pressing the composition into a solid layer possessing dimensions that correspond to the internal dimensions of the compartment space adjacent to a passageway. In another embodiment, the drug and other drug composition forming ingredients and a solvent are mixed into a solid, or semi-solid, by conventional methods such as ball-milling, calendering, stirring or roll-milling, and then pressed into a preselected, layer-forming shape.

In the embodiment presented above, the composition or a layer of the composition comprising a hydrogel osmopolymer and an optional osmagent is placed in contact with the layer comprising the drug, and the two layers comprising the layers are surrounded with a semipermeable membrane. The layering of the drug composition and the hydrogel push-layer and optional osmagent composition can be accomplished by using a conventional two-layer tablet press technique. The wall can be deposited through the molding, spraying, or dipping of pressed shapes with semi-permeable membrane forming materials. Another technique that can be used for applying the semi-permeable membrane is the air-suspension coating procedure. This procedure consists in suspending and tumbling the two layers in a current of air until the semi-permeable membrane forming composition surrounds the layers. Alternatively, the semi-permeable membrane may be formed through a pan coating process, wherein the pressed shapes are tumbled in a pan while the semi-permeable membrane forming composition is sprayed onto said shapes. Manufacturing procedures are described in Modern Plastics Encyclopedia, Vol. 46, pp. 62-70 (1969); and in Pharmaceutical Sciences, by Remington, 14th Ed., pp. 1626-1648 (1970), published by Mack Publishing Co., Easton, Pa. The dosage form can be manufactured by following the teaching in U.S. Pat. Nos. 4,327,725; 4,612,008; 4,783,337; 4,863,456; and 4,902,514.

Exemplary solvents suitable for manufacturing the wall, the composition layers and, the dosage form include inert inorganic and organic solvents that do not adversely harm the materials, the wall, the layer, the composition and the drug wall. Any flux enhancers in the wall composition are first dissolved in the solvent under stirring with or without the aid of heat. Such solvents may be aqueous, organic, or mixtures thereof. After complete dissolution of the flux enhancers, the cellulosic component, cellulose acetate butyrate, for example, of the wall-forming material is added and stirring is continued until both components are in solution. The membrane may be applied onto the core by using a Wurster coater, pan coater or any other coating equipment. Alternatively a blend of the coating materials may also be directly compressed onto the said core. A desirable thickness of the semi-permeable membrane is approximately 4.6 mils.

Dosage forms in accordance with the embodiments depicted herein may be manufactured by standard techniques. For example, the dosage form may be manufactured by the wet granulation technique. In the wet granulation technique, the drug and carrier are blended using an organic solvent, such as denatured anhydrous ethanol, as the granulation fluid. The remaining ingredients can be dissolved in a portion of the granulation fluid, such as the solvent described above, and this latter prepared wet blend is slowly added to the drug blend with continual mixing in the blender. The granulating fluid is added until a wet blend is produced, which wet mass blend is then forced through a predetermined screen onto oven trays. The blend is dried for 18 to 24 hours at 24° C. to 35° C. in a forced-air oven. The dried granules are then sized. Next, magnesium stearate, or another suitable lubricant, is added to the drug granulation, and the granulation is put into milling jars and mixed on a jar mill for 10 minutes. The composition is pressed into a layer, for example, in a Manesty® press or a Korsch LCT press. For a trilayered core, granules or powders of the drug layer compositions and push layer composition are sequentially placed in an appropriately-sized die with intermediate compression steps being applied to each of the first two layers, followed by a final compression step after the last layer is added to the die to form the trilayered core. The intermediate compression typically takes place under a force of about 50-100 newtons. Final stage compression typically takes place at a force of 3500 newtons or greater, often 3500-5000 newtons. The compressed cores are fed to a dry coater press, e.g., Kilian® Dry Coater press, and subsequently coated with the semi-permeable membrane materials as described above.

In another embodiment, the drug and other ingredients comprising the drug layer are blended and pressed into a solid layer. The layer possesses dimensions that correspond to the internal dimensions of the area the layer is to occupy in the dosage form, and it also possesses dimensions corresponding to the push layer, if included, for forming a contacting arrangement therewith. The drug and other ingredients can also be blended with a solvent and mixed into a solid or semisolid form by conventional methods, such as ballmilling, calendering, stirring or rollmilling, and then pressed into a preselected shape. Next, if included, a layer of osmopolymer composition is placed in contact with the layer of drug in a like manner. The layering of the drug formulation and the osmopolymer layer can be fabricated by conventional two-layer press techniques. An analogous procedure may be followed for the preparation of the trilayered core. The compressed cores then may be coated with the inner wall material and the semipermeable wall material as described above.

Another manufacturing process that can be used comprises blending the powdered ingredients for each layer in a fluid bed granulator. After the powdered ingredients are dry blended in the granulator, a granulating fluid, for example, poly(vinylpyrrolidone) in water, is sprayed onto the powders. The coated powders are then dried in the granulator. This process granulates all the ingredients present therein while adding the granulating fluid. After the granules are dried, a lubricant, such as stearic acid or magnesium stearate, is mixed into the granulation using a blender e.g., V-blender or tote blender. The granules are then pressed in the manner described above.

Exemplary solvents suitable for manufacturing the dosage form components comprise aqueous or inert organic solvents that do not adversely harm the materials used in the system. The solvents broadly include members selected from the group consisting of aqueous solvents, alcohols, ketones, esters, ethers, aliphatic hydrocarbons, halogenated solvents, cycloaliphatics, aromatics, heterocyclic solvents and mixtures thereof. Typical solvents include acetone, diacetone alcohol, methanol, ethanol, isopropyl alcohol, butyl alcohol, methyl acetate, ethyl acetate, isopropyl acetate, n-butyl acetate, methyl isobutyl ketone, methyl propyl ketone, n-hexane, n-heptane, ethylene glycol monoethyl ether, ethylene glycol monoethyl acetate, methylene dichloride, ethylene dichloride, propylene dichloride, carbon tetrachloride nitroethane, nitropropane tetrachloroethane, ethyl ether, isopropyl ether, cyclohexane, cyclooctane, benzene, toluene, naphtha, 1,4-dioxane, tetrahydrofuran, diglyme, water, aqueous solvents containing inorganic salts such as sodium chloride, calcium chloride, and the like, and mixtures thereof such as acetone and water, acetone and methanol, acetone and ethyl alcohol, methylene dichloride and methanol, and ethylene dichloride and methanol.

Following coating, the coated osmotic cores of the present invention are dried. In an embodiment, the coated osmotic cores are dried in a pan coater, thus saving a unit operation as compared to moving the coated osmotic cores to a separate drier. In another embodiment, the coated osmotic cores are moved to a separate drier and are dried. Varying the settings of the pan coater or the drier to obtain desired drying conditions for specific coated osmotic dosage forms and batch sizes, etc. is conventionally known.

While there has been described and pointed out features and advantages of the invention, as applied to present embodiments, those skilled in the medical art will appreciate that various modifications, changes, additions, and omissions in the method described in the specification can be made without departing from the spirit of the invention.

### EXAMPLES

The following examples are meant to be illustrative of the claimed invention and not limiting in any manner.

### Example 1

### Oxybutynin Osmotic Composition

A therapeutic oxybutynin composition provided by the invention is prepared as follows: first, 103 grams of oxybutynin hydrochloride is dissolved in 1200 milliliters of anhydrous ethanol. Separately, 2,280 g of polyethylene oxide of 200,000 molecular weight, 150 g of hydroxypropylmethylcellulose of 9,200 molecular weight and 450 g of sodium chloride are dry blended in a conventional blender for 10 minutes to yield a homogenous blend. Next, the oxybutynin ethanol solution is added slowly, with the mixer continuously blending until all the solution is added to the three component dry blend, with the mixing continuing for another 8 to 10 minutes. The blended wet composition is passed through a 16 mesh screen and dried over night at a room temperature of 72. degree. F. (22.2. degree.). Then, the dry granules are passed through a 20 mesh screen and 18 g of magnesium stearate are added and all the ingredients blended again for 5 minutes. The fresh granules are ready for formulation into a therapeutic oxybutynin composition. The therapeutic composition comprises 3.4 wt % oxybutynin hydrochloride, 76 wt % polyethylene oxide of 200,000 molecular weight, 5 wt % of hydroxypropylmethylcellulose of 9,200 molecular weight, 15 wt % sodium chloride, and 0.6 wt % magesium stearate.

### Example 2

### Push Layer Composition

An osmopolymer hydrogel composition provided by the invention is prepared as follows: first 1274 g of pharmaceutically acceptable polyethylene oxide of approximately 7,500,000 molecular weight, 600 g of sodium chloride, and 20 g ferric oxide are separately screened through a 40 mesh screen. Then, all the screened ingredients are mixed with 100 g of hydroxypropylmethylcellulose of 11,200 molecular weight to produce a homogenous blend. Next, 300 ml of denatured anhydrous alcohol is added slowly to the blend with continuous mixing for 5 minutes. Then, 1.6 g of butylated hydroxytoluene is added followed by more blending with 5 g of magnesium stearate being added with 5 minutes of blending to yield a homogenous blend. The freshly prepared granulation is passed through a 20 mesh screen and allowed to dry for 20 hours at 22.2. degree. C. The final composition comprises 63.67 wt % of the polyethylene oxide, 30 wt % of sodium chloride, 1 wt % of ferric oxide, 5 mg of hydroxypropylmethylcellulose, 0.08 wt % of butylated hydroxytoluene, and 0.25 mg of magnesium stearate.

### Example 3

### Tablets Comprising Oxybutynin

The therapeutic oxybutynin composition of Example 1 and the osmopolymer hydrogel composition of Example 2 are made into a bilayer tablet as follows: first, 147 mg of the oxybutynin composition is added to a punch die set, and tamped, then, 98 mg of the hydrogel composition is added and the two layers are compressed under a pressure head of 1.0 ton (1000 kg) into a 11/32 inch (0.873 cm) diameter, to form a contacting intimate bilayered tablet.

### Example 4

### Coated Osmotic Cores

The bilayered tablet of Example 3 is manufactured into a delivery device as follows: first, a semipermeable wall-forming composition is prepared comprising 80 wt % cellulose acetate butyrate 171-15 having a 29.69% acetyl content and 16.88% butyryl content wt and 20% poloxamer 188 by dissolving the ingredients in a co-solvent comprising acetone and water in 99.5:0.5 wt:wt composition to make a 5% solid solution. The wall-forming composition is sprayed onto and around the bilayered core to provide a 17.8 mg semipermeable wall. The coating is accomplished first using a 12″ LDCS coater and further coating studies are carried out using 24″ Vector Hi-Coater. Next, the semipermeable walled bilayered tablet is laser drilled through the semipermeable wall to provide a 25 mil (0.64 mm) to contact the oxybutynin layer with the exterior of the delivery device.

The samples coated are dried for 3 and 7 days at 45. degree. C. and 45% relative humidity and tested for residual solvent content using Gas chromatography.

The coated osmotic cores provided by this manufacture provide 3.4 wt % oxybutynin hydrochloride, 76 wt % polyethylene oxide of 200,000 molecular weight, 5 wt % hydroxypropylmethylcellulose of 9,200 molecular weight, 0.6 wt % magnesium stearate, and 15 wt % sodium chloride in the therapeutic oxybutynin composition. The osmopolymer, hydrogel push composition comprises 63.67 wt % polyethylene oxide of 7,500,000 molecular weight, 30 wt % sodium chloride, 1 wt % ferric chloride, 5 wt % hydroxypropylmethylecellulose of 9,200 molecular weight, 0.08 wt % butylated hydroxytoluene, and 0.25 wt % magnesium stearate. The semipermeable wall comprises 80 wt % cellulose acetate butyrate comprising 29.9% acetyl content, and 20 wt % poloxamer 188. The delivery device comprises an exit passage of 25 mils (0.64 mm) and it has a mean release rate of 0.260 mg/hr for 23.8 hours. The semipermeable wall provides substantial protection from photo (light) degradation of the oxybutynin in the delivery device.

### Example 5

Uncoated osmotic oxybutynin bilayer production cores (from Ditropan XL®) were obtained for a coating study. Two coating formulations, CA 398-10:PEG 3350 (99:1) and CAB 171-15 PG:Poloxamer 188 were selected.

The coating was performed on a 12″ coating scale using an LDCS-HL 20/30 Model coater (Vector Corp.). The compositions and results are shown in Table 1. The operating parameters were as follows: Load weight:˜0.1 kg; Gun to bed distance:2¾″; Inlet Temp:40C; Exhaust temp:25 C; Pan (rpm):28; Pump run (%):36; Soution spray rate: 23 g/min. Results are shown in Table 1.

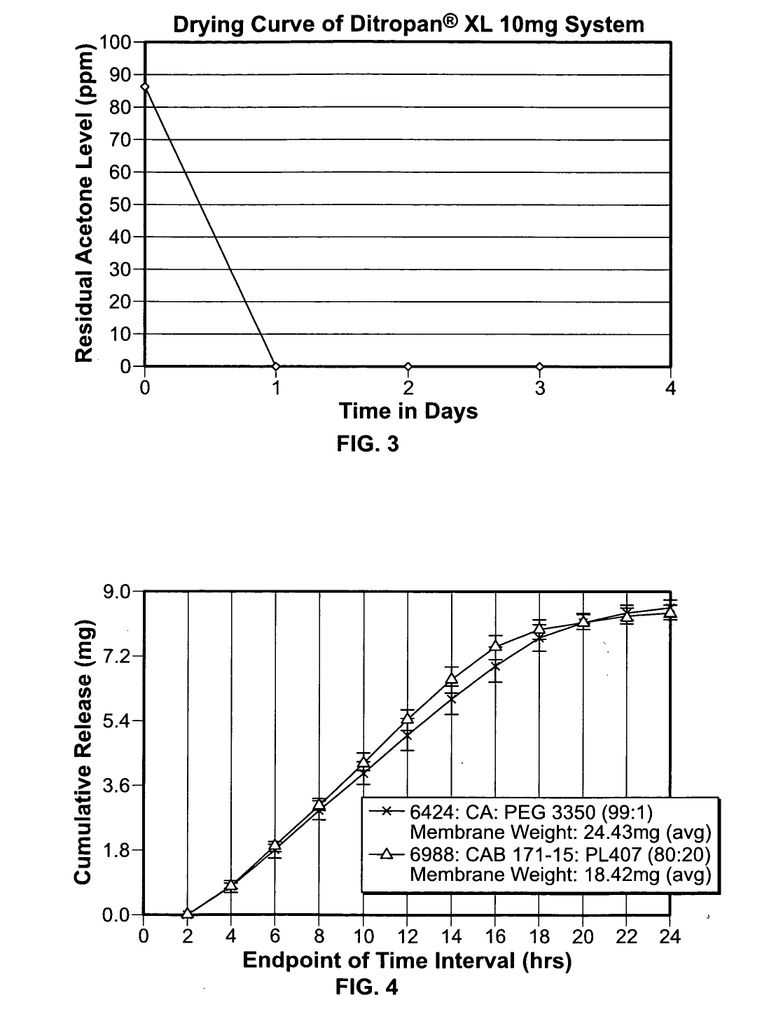

The CAB 171-15PG:poloxamer 188 (80:20) composition was further evaluated at 24″ coating scale on a Vector Hi-Coater HCT60. The results are shown in Table 2 and FIG. 3. The operating parameters were as follows: Load weight:˜10.1 Kg; Gun to bed distance:5.5″; Atomization airflow (SLPM):80; Gun air Flow (SLPM):30; Inlet Temp:45; Exhaust temp: as needed usually 33 C; Pan (rpm):11; Pan pressure(in of water): −2.0; Pump run (%):100; Solution spray rate:˜166 g/min.

Table 2 and FIG. 3 provides a system comparison between the two membrane formulations.

Release rate studies were undertaken with the coated osmotic cores. The release rate was determined using USP type VII apparatus. The medium used was artificial gastric fluid (pH 1.2) followed by HPLC analysis for estimation of the sample. Table 3 and FIG. 4 show the results of these studies.

TABLE 1
Residual Solvent Data on Ditropan XL 10 mg coated in 12″ LDCS coater.

| | | Drying | | Average |
|---|---|---|---|---|
| | | Time in | Average | Residual |
| | | Days at | Membrane | Acetone |
| | Coating | 45° C./ | Weight | Content |
| Membrane Composition | ID | 45% RH | (mg) | (ppm) |
| CAB 171-15 PG:Poloxamer | Run I | 3 | 17.1 | 0 |
| 188 (80:20) | | 7 | 17.1 | 0 |
| CAB 171-15 PG:Poloxamer | Run II | 3 | 21.1 | 0 |
| 188 (75:25) | | 7 | 21.1 | 0 |
| CAB 171-15 PG:Poloxamer | Run III | 3 | 17.4 | 0 |
| 338 (80:20) | | 7 | 17.4 | 0 |
| CAB 171-15 PG:PEG | Run IV | 3 | 17.1 | 0 |
| 3350 (80:20) | | 7 | 17.1 | 0 |
| CA:PEG3350 (99:1) | Run 1 | 3 | 24.5 | 1,165 |
| | | 7 | 24.5 | 415 |

TABLE 2
Residual Solvent Data on Ditropan XL 10 mg coated in 24″ LDCS coater

| Drying Time in Days at | Average Membrane | Average Residual |
|---|---|---|
| 45° C./45% RH | Weight (mg) | Acetone Content (ppm) |
| 0 | 17.85 | 86.30 |
| 1 | 17.85 | 0 |
| 2 | 17.85 | 0 |
| 3 | 17.85 | 0 |

TABLE 3
System Functionality Summary comparison of CAB 171-15
PG (Low K) and CA 398-membranes (Standard) Coated onto Ditropan XL 10 mg Cores

| | Coating ID | | | |
|---|---|---|---|---|
| | 24″ | 12″ | 12″ | 24″ |
| | Run1 | Run2 | Run3 | Run4 |
| Membrane | CA:PEG | CAB 171-15 | CAB 171-15 | CAB 171-15 |
| Components | 3350 | PG:Poloxamer 188 | PG:Poloxamer | PG:Poloxamer 188 |
| | (99:1) | (80:20) | 188 (80:20) | (80:20) |
| Drying Time, day | 3 | 3 | 7 | 5 |
| Average | 24.43 | 16.78 | 16.98 | 18.42 |
| Membrane | | | | |
| Weight, mg | | | | |
| Average Release | 0.52 | 0.55 | 0.56 | 0.57 |
| Rate, mg/hr | | | | |
| R.R * MW | 12.70 | 9.23 | 9.50 | 10.49 |
| Start-Up Time, hr | 2.5 | 3.1 | 3.3 | 2.6 |
| % Residual Drug | 16.38 | 16.76 | 16.54 | 15.38 |
| Content | | | | |
| Within System | 3.9 | 5.9 | 4.1 | 3.4 |
| Variability, % | | | | |
| Between- | 4.8 | 8.4 | 9.4 | 3.4 |
| Systems | | | | |
| Variability, % | | | | |
| T50, hr | 12.2 | 12.3 | 12.2 | 11.3 |

### Example 6

### WG-1 Granulation

6 gms of Topiramate, 5.04 gm of polyethylene oxide N-10, 0.6 gm Povidone (PVP K29-32) and 7.80 g of Poloxamer 407 are accurately weighed into a beaker. The mixture is granulated using an aeromixer by adding ethyl alcohol as a granulating fluid. The granulation is dried overnight at room temperature and sieved through a 16 mesh sieve. The sieved granulation is weighed and transferred to a glass jar. 2% w/w of stearic acid (previously sieved through 40 mesh sieve) and 0.05% w/w of BHT(previously sieved through 40 mesh sieve) is added and blended by placing the jar on a roller mill for 10 minutes. 0.75% w/w Magnesium stearate (previously sieved through 40 mesh sieve) is added into the jar and blended for 30 seconds.

### Example 7

### WG-2 Granulation

8 gm of Topiramate, 0.43 gm of polyethylene oxide N-10, 0.6 gm Povidone (PVP K29-32), 0.02 gm of Ferric Oxide (black) and 10.40 gm of Poloxamer 407 are accurately weighed into a beaker. The mixture is granulated using an aeromixer by adding ethyl alcohol as a granulating fluid. The granulation is dried overnight at room temperature and sieved through a 16 mesh sieve. The sieved granulation is weighed and transferred to a glass jar. 2% w/w of stearic acid (previously sieved through 40 mesh sieve) and 0.02% w/w of BHT (previously sieved through 40 mesh sieve) is added and blended by placing the jar on a roller mill for 10 minutes. 0.75% w/w Magnesium stearate (previously sieved through 40 mesh sieve) is added into the jar and blended for 30 seconds.

### Example 8

### Push Layer Granulation

The osmopolymer hydrogel composition provided by the invention is prepared as follows: first 1274 g of pharmaceutically acceptable polyethylene oxide comprising a 7,500,000 molecular weight, 600 g of sodium chloride, and 20 g ferric oxide are separately screened through a 40 mesh screen. Then, all the screened ingredients are mixed with 100 g of hydroxypropylmethylcellulose of 11,200 molecular weight to produce a homogenous blend. Next, 300 ml of denatured anhydrous alcohol is added slowly to the blend with continuous mixing for 5 minutes. Then, 1.6 g of butylated hydroxytoluene is added followed by more blending with 5 g of magnesium stearate added with 5 minutes of blending to yield a homogenous blend. The freshly prepared granulation is passed through a 20 mesh screen and allowed to dry for 20 hours at 22.2. degree. C. The final composition comprises 63.67 wt % of the polyethylene oxide, 30 wt % of sodium chloride, 1 wt % of ferric oxide, 5 mg of hydroxypropylmethylcellulose, 0.08 wt % of butylated hydroxytoluene, and 0.25 mg of magnesium stearate.

### Example 9

### Coated Osmotic Cores

Trilayer cores consisting of 53 mg WG-1 granulation from Example 6, 73 mg WG-2 granulation from Example 7, along with 93 mg push layer granulation from Example 8 are hand compressed on a Carver press using 3/16″ LCT tooling with 0.25 ton compression force. These trilayer cores are subcoated with (70:30) [HPC:PVP(K29-32)] in ethanol (8% solids) using a Vector Hi-Coater to a target subcoat weight of 21.59 mg. The subcoated cores are further membrane coated with CAB 171-15: Poloxamer 188 (80:20) in 99.5:0.5 acetone:water (5% solids) to a membrane weight of 20.2 mg using a Vector Hi-Coater. The membrane coated tablets are drilled with a 40 mil orifice and dried for 2 days at 40° C./Ambient humidity in a VWR oven.

### Example 10

### Topiramate Zero-Order Cores

Drug Layer Granulation: The drug layer granulation was manufactured at the medium scale on the Glaft GPCG-30 fluid bed granulator while the push layer granulation was manufactured at the at 120 Kg scale on Large fluid bed granulator. The exact composition of the drug and push layers are illustrated in Table 4.

Drug layer Granulation: The drug granulation was manufactured as two sublots. Sublot A was manufactured by charging 2.88 Kg of Topiramate and 958 g of Polethylene oxide, N-80, 200K and 4.982 kg of micronized Poloxamer 407 into Glatt GPCG-30 fluid bed granulator. A binder solution consisting of 10% (w/w) of poloxamer 407 and purified water was prepared by dissolving the poloxamer in water. A 15% (w/w) solution of the povidone binder solution was prepared by dissolving the povidone in purified water. The inlet temperature was controlled between 30-32° C. and the airflow was adjusted on the Glatt to maintain fluidization and 3.785 Kg of the poloxamer binder solution was sprayed, followed by 3.33 Kg of Povidone binder solution. The granulation was dried to a target moisture content of 0.5%. The Sublot B was manufactured in a similar manner. Both the sublots were then sized by passing the granulation through a granumill fitted with 7 mesh screen. The sublots were then blended in a rotational mixer for 10 minutes. The BHT and Stearic acid were passed through a 40 mesh screen. The sieved BHT was blended into the granulation by rotating the tote using a tote tumbler for 5 minutes. The stearic acid was then blended into the granulation by rotating the tote tumbler for 5 minutes. The Magnesium state was then added and blended for 30 seconds.

Push layer granulation: The ferric oxide and the sodium chloride was milled seperatley using Quadro comill fitted with a 21 mesh screen and collected in to separate drums. 80.4 Kg od Polyethylene oxide 303, 37.5 Kg of sized sodium chloride and 0.5 kg of Ferric oxide was charged into the tote and loaded into the Glatt fluid bed granulator. A binder solution consisting of 13% (w/w) of povidone in purified water was used as a binding agent. The inlet temperature was controlled between 43-47° C. and the airflow was adjusted on the Glatt to maintain fluidization and 6.25 Kg of the binder solution was sprayed. After the binder solution was sprayed the granulation was dried to a target moisture content of <1%. The granulation was then milled using a granumill fitted with a 7 Mesh screen and collected into a tote. The BHT and Stearic acid were passed through a 40 mesh screen. The sieved BHT was blended into the granulation by rotating the tote using a tote tumbler for 10 minutes. The stearic acid was then blended into the granulation by rotating the tote tumbler for 1 minute.

Core Compression: The Cores were compressed on the Korsch Multilayer Press using 33 stations of 15/64″ deep concave tooling. All in-process test results were well within the acceptance limits. The target first layer weights for the drug and push layers are listed in Table 1.

Membrane Coating: The systems were coated with either a standard CA:PEG membrane or the new CAB:PL407 membrane using 24″ Vector Hi-Coater.

Drilling: After coating, the tablets were drilled using Servo drill with a 1.15-mm (45 mil) orifice on the drug layer dome of the system

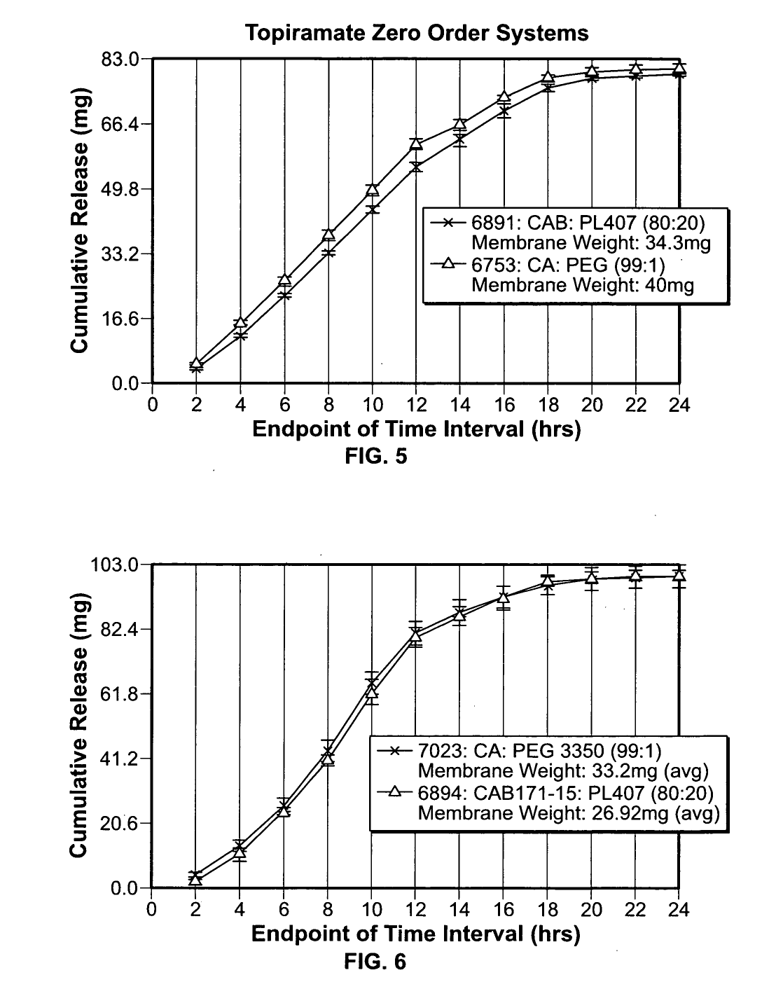

Drilled cores were dried in the Hotpack oven for at 40° C. and ambient humidity. Dried systems were sampled for residual solvent testing and tested for release rate. The systems details are listed in Table 4 while system performance is provided in Tables 6, 7, 8 and depicted in FIG. 5.

TABLE 4
Formulation of OROS ® Topiramate 100 mg (Zero Order Profile)

| | | Quantity |
|---|---|---|
| | | (mg) per |
| Description | Target % | tablet |
| Drug Layer, 278 mg | | |
| Topiramate | 28.80 | 80.06 |
| Polyethylene Oxide, NF, N-80, 200K, TG, LEO | 9.58 | 26.63 |
| Poloxamer 407, NF (Micronized) | 53.60 | 149.01 |
| Povidone, USP, Ph Eur, (K29-32) | 5.00 | 13.90 |
| Stearic Acid, NF, Ph Eur, (Powder) | 2.00 | 5.56 |
| Magnesium Stearate, NF, Ph Eur | 1.00 | 2.78 |
| BHT, FCC, Ph Eur, (Milled) | 0.02 | 0.06 |
| Purified Water, USP, Ph Eur, (In Containers) | — | Trace |
| Push Layer, 185 mg | | |
| Granulation, OROS ® Push Layer, 30% NaCl | 100 | 185.00 |
| Polyethylene Oxide, NF, 303, 7000K, TG, LEO | 64.3 | |
| Sodium Chloride, USP, Ph.Eur, (powder) | 30.0 | |
| Povidone, USP, Ph.Eur, (K29-32) | 5.0 | |
| Ferric oxide, NF, (Red) | 0.4 | |
| Stearic Acid, NF, (Powder) | 0.25 | |
| BHT, FCC, Ph.Eur, (Milled) | 0.05 | |
| Purifies Water, USP, Ph.Eur (As required) | — | Trace |
| Membrane Coating | | |
| Cellulose Acetate Butyrate, CAB 171-15PG | 80.00 | |
| Poloxamer 188, NF, Ph Eur | 20.00 | |
| Acetone, NF, (Bulk) | — | Trace |
| OR | | |
| Cellulose Acetate, 398-10, NF | 99 | |
| Polyethylene Glycol 3350 | 1 | |
| Acetone, NF, (Bulk) | | Trace |

### Example 11

### Topiramate Ascending Cores

Drug Layer 1 Granulation

The drug layer granulation 1 was manufactured by charging 3.0 Kg of Topiramate and 2.520 g of Polethylene oxide, N-80, 200K and 3.630 kg of micronized Poloxamer 407 into Glatt GPCG-30 fluid bed granulator. A binder solution consisting of 10% (w/w) of poloxamer 407 and purified water was prepared by dissolving the poloxamer in water. A 15% (w/w) solution of the povidone binder solution was prepared by dissolving the povidone in purified water. The inlet temperature was controlled between 28-32° C. and the airflow was adjusted on the Glatt to maintain fluidization and 2.7 Kg of the poloxamer binder solution was sprayed, followed by 2.0 Kg of Povidone binder solution. The granulation was dried to a target moisture content of 0.5%. The granulation was sized by passing the granulation through a granumill fitted with 7 mesh screen. The BHT and Stearic acid were passed through a 40 mesh screen. The sieved BHT was blended into the granulation by rotating the tote using a tote tumbler for 5 minutes. The stearic acid was then blended into the granulation by rotating the tote tumbler for 5 minutes. The Magnesium state was then added and blended for 30 seconds.

Drug Layer 2 Granulation

The drug layer granulation 2 was manufactured by charging 4.0 kg of Topiramate and 213 g of Polethylene oxide, N-80, 200K, 4.840 kg of micronized Poloxamer 407 and 10 gm of black iron oxide (sieved through 10 mesh sieve) into Glatt GPCG-30 fluid bed granulator. A binder solution consisting of 10% (w/w) of poloxamer 407 and purified water was prepared by dissolving the poloxamer in water. A 15% (w/w) solution of the povidone binder solution was prepared by dissolving the povidone in purified water. The inlet temperature was controlled between 28-32° C. and the airflow was adjusted on the Glatt to maintain fluidization and 3.6 Kg of the poloxamer binder solution was sprayed, followed by 2.0 Kg of Povidone binder solution. The granulation was dried to a target moisture content of 0.5%. The granulation was sized by passing the granulation through a granumill fitted with 7 mesh screen. The BHT and Stearic acid were passed through a 40 mesh screen. The sieved BHT was blended into the granulation by rotating the tote using a tote tumbler for 5 minutes. The stearic acid was then blended into the granulation by rotating the tote tumbler for 5 minutes. The Magnesium state was then added and blended for 30 seconds.

Two drug layer granulations were manufactured separately in the 45 L-bowl of the Glatt GPCG-30 fluid bed granulator. The vacuum and filter bags were weighed before and after granulation in order to determine process losses and accountability. The two binder solutions were prepared, one of which consisted of Poloxamer 407 in purified water and the other of PVP in purified water. The topiramate, polyethylene oxide, and the remaining amount of Poloxamer 407 (and addition of the black ferric oxide for drug layer 2) were charged to the bowl dry. While granulating, the required amount of binder solution was metered into the granulator. One-fourteenth of the Poloxamer 407 in the formulation was sprayed onto the granulation bed followed by all of the required PVP in the formulation. Process air volume was adjusted on an as-needed basis during the run to maintain proper fluidization. After the binder solutions were applied, the granulation was dried to the target moisture content of 0.5%, with acceptable range of 0.2-0.8%.

After drying, the granulation was sized through a 7.2 -mesh screen using the Fluid Air Granumill. Then, screened butylated hydroxytoluene (BHT) and sized stearic acid and magnesium stearate were blended into the granulation using the Gemco Slant Cone blender.

Push Granulation

The ferric oxide and the sodium chloride was milled seperatley using Quadro comill fitted with a 21 mesh screen and collected in to separate drums. 80.4 Kg od Polyethylene oxide 303, 37.5 Kg of sized sodium chloride and 0.5 kg of Ferric oxide was charged into the tote and loaded into the Glatt fluid bed granulator. A binder solution consisting of 13% (w/w) of povidone in purified water was used as a binding agent. The inlet temperature was controlled between 43-47° C. and the airflow was adjusted on the Glatt to maintain fluidization and 6.25 Kg of the binder solution was sprayed. After the binder solution was sprayed the granulation was dried to a target moisture content of <1%. The granulation was then milled using a granumill fitted with a 7 Mesh screen and collected into a tote. The BHT and Stearic acid were passed through a 40 mesh screen. The sieved BHT was blended into the granulation by rotating the tote using a tote tumbler for 10 minutes. The stearic acid was then blended into the granulation by rotating the tote tumbler for 1 minute.

Core Compression

The cores were compressed with a drug-to-push ratio of 1.4 on the Korsch Multi-layer press using ¼″ deep concave tooling. The press was set up with 33 stations and run at a speed of 13 rpm. Target tamping forces of 100 N on both drug layers and final compression force of 3000 N were applied to compress the trilayer cores.

Membrane Coating: The systems were coated with either a standard CA:PEG membrane or the new CAB:PL407 membrane using 24″ Vector Hi-Coater. The formulation is listed in Table 1.

Drilling: After coating, the tablets were drilled using Servo drill with a 1.0 mm (40 mil) orifice on the drug layer dome of the system

Drying

Drilled cores were dried in the Hotpack oven for at 40° C. and ambient humidity. Dried systems were sampled for residual solvent testing and tested for release rate. The systems details are listed in Table 3 and Table 4 and the release is depicted in FIG. 6.

TABLE 5
Formulation of OROS ® Topiramate 100 mg (Ascending Profile)

| | | Quantity |
|---|---|---|
| Description | Target % | (mg) |
| Drug Layer 1 Granulation, 120 mg | | |
| Topiramate | 30.00 | 36.00 |
| Polyethylene Oxide, NF, N-80, 200K, TG, LEO | 25.20 | 30.24 |
| Poloxamer 407, NF (Micronized) | 39.00 | 46.80 |
| Povidone, USP, Ph Eur, (K29-32) | 3.00 | 3.60 |
| Stearic Acid, NF, Ph Eur, (Powder) | 2.00 | 2.40 |
| Magnesium Stearate, NF, Ph Eur | 0.75 | 0.90 |
| BHT, FCC, Ph Eur, (Milled) | 0.05 | 0.06 |
| Drug Layer 2 Granulation, 160 mg | | |
| Topiramate | 40.00 | 64.00 |
| Polyethylene Oxide, NF, N-80, 200K, TG, LEO | 2.13 | 3.408 |
| Poloxamer 407, NF (Micronized) | 52.00 | 83.20 |
| Iron Oxide Black | 0.10 | 0.16 |
| Povidone, USP, Ph Eur, (K29-32) | 3.00 | 4.80 |
| Stearic Acid, NF, Ph Eur, (Powder) | 2.00 | 3.20 |
| Magnesium Stearate, NF, Ph Eur | 0.75 | 1.20 |
| BHT, FCC, Ph Eur, (Milled) | 0.02 | 0.032 |
| Push Layer Granulation, 200 mg | | |
| Polyethylene Oxide, NF, 303, 7000K, TG, LEO | 64.30 | 128.6 |
| Sodium Chloride, USP, Ph Eur, (Powder) | 30.00 | 60.00 |
| Povidone, USP, Ph Eur, (K29-32) | 5.00 | 10.00 |
| Ferric Oxide, NF, (Red) | 0.40 | 0.8 |
| Stearic Acid, NF, Ph Eur, (Powder) | 0.25 | 0.5 |
| BHT, FCC, Ph Eur, (Milled) | 0.05 | 0.1 |
| Membrane Coat | | |
| Cellulose Acetate Butyrate, CAB 171-15PG | 80.00 | |
| Poloxamer 188, NF, Ph Eur | 20.00 | |
| Acetone, NF, (Bulk) | | Trace |
| OR | | |
| Cellulose Acetate, 398-10, NF | 99 | |
| Polyethylene Glycol 3350 | 1 | |
| Acetone, NF, (Bulk) | | Trace |

TABLE 6
System Functionality Summary comparison of CAB 171-15
PG (Low K) and CA 398-membranes (Standard)
Coated onto AP-42 zero-order Cores

| Coating ID | CAPEG-2-ZO | CABPX-T |
|---|---|---|
| Membrane Components | CA 398-10:PEG | CAB 171-15:Poloxamer |
| | (99:1) | 188 (80:20) |
| Drying Time, day | 7 | 5 |
| Average Membrane | 42.9 | 34.3 |
| Weight, mg | | |
| Average Release Rate, | 5.69 | 5.39 |
| mg/hr | | |
| R.R * MW | 227.6 | 184.9 |
| Start-Up Time, hr | 1.2 | 1.7 |
| % Residual Drug Content | 0.98 | 1.72 |
| Within System Variability, | 2.5 | 4.0 |
| % | | |
| Between-Systems | 1.5 | 1.9 |
| Variability, % | | |
| T90, hr | 16.1 | 16.9 |

TABLE 7
Residual Solvent Data for AP-42 Topiramate Coated with
CAB 171-15 PG:Poloxamer 188 (80:20)

| | Average | Drying time in days | Average Residual |
|---|---|---|---|
| | Membrane | at 37° C./Ambient | Solvent content |
| Sample Id | Weight (mg) | humidity | (ppm) |
| AP-42 Zero Order | 33 | 0 | 4882.62 |
| AP-42 Zero Order | 33 | 1 | 799.83 |
| AP-42 Zero Order | 33 | 2 | 233.83 |
| AP-42 Zero Order | 33 | 3 | 84.94 |
| AP-42 Ascending | 34.5 | 0 | 4780.55 |
| AP-42 Ascending | 34.5 | 1 | 605.35 |
| AP-42 Ascending | 34.5 | 2 | 163.15 |

TABLE 8
Residual Solvent Data for AP-42 Topiramate Coated with
CA 398-10:PEG 3350 (99:1)

| | Average | Drying time in | |
|---|---|---|---|
| | Membrane | days at | Average Residual |
| | Weight | 40° C./Ambient | Solvent content |
| Sample Id | (mg) | humidity | (ppm) |
| AP-42 Zero Order | 40 | 0 | 6925.85 |
| AP-42 Zero Order | 40 | 3 | 3269.63 |
| AP-42 Zero Order | 40 | 5 | 3082.33 |
| AP-42 Zero Order | 40 | 7 | 2667.28 |

What is claimed is:

1. A method comprising:
providing an osmotic core;
coating the osmotic core with a coating composition that comprises cellulose acetate butyrate;
drying the coated osmotic core for a maximum period of about 36 hours;
wherein the coated core is dried to an average residual solvent content of less than about 1000 parts per million.

2. The method of claim 1, wherein the coating composition further comprises from about 0.01% to about 40% by weight or more of a flux regulator, based on total solids in the coating composition.

3. The method of claim 2, wherein the coating composition further comprises from about 10 wt % to about 30 wt % of a flux regulator, based on total solids in the coating composition.

4. The method of claim 1, wherein the coated osmotic core is dried for a maximum period of about 24 hours;

5. The method of claim 1, wherein the coated core is dried to an average residual solvent content of less than about 500 parts per million.

6. The method of claim 1, wherein the osmotic core comprises a trilayer osmotic core.

7. A dosage form comprising the coated osmotic core of claim 1

8. A method comprising:
providing an osmotic core;
coating the osmotic core with cellulose acetate butyrate;
drying the coated osmotic core for a maximum period of about 24 hours;
administering the coated osmotic core to a patient;
wherein the coated core is dried to an average residual solvent content of less than about 1000 parts per million.

9. The method of claim 8, wherein the coating composition further comprises from about 0.01% to about 40% by weight or more of a flux regulator, based on total solids in the coating composition.

10. The method of claim 9, wherein the coating composition further comprises from about 10 wt % to about 30 wt % of a flux regulator, based on total solids in the coating composition.

11. The method of claim 8, wherein the coated osmotic core is dried for a maximum period of about 24 hours;

12. The method of claim 8, wherein the coated core is dried to an average residual solvent content of less than about 500 parts per million.

13. The method of claim 8, wherein the osmotic core comprises a trilayer osmotic core.

14. The method of claim 8, wherein the osmotic core comprises an osmagent.

15. The method of claim 8, wherein the coated osmotic core is orally administered to a patient.
